(19) United States
(12) Patent Application Publication
(10) Pub. No.: US 2005/0142225 A1
(43) Pub. Date: Jun. 30, 2005

(54) Stabilised pharmaceutical compositions on the basis of polyoxyethlated castor oil and method for manufacturing the same

(75) Inventors: Vladimir Kysilka, Brno (CZ); Libuse Zatloukalova, Brno (CZ); Borek Zaludek, Brno (CZ); Karel Pospisilik, Brno (CZ); Mario Cigankova, Brno (CZ); Martin Kubat, Brno (CZ); Ales Cirkva, Brno (CZ)

Correspondence Address: DARBY & DARBY P.C., P. O. BOX 5257, NEW YORK, NY, 10150-5257, US

(73) Assignee: Pliva-Lachema A.S., Brno (CZ)

(21) Appl. No.: 10/513,751

(22) PCT Filed: May 16, 2003
(86) PCT No.: PCT/EP03/05153

(51) Int. Cl. A61K 35/78; A61K 31/4745; A61K 31/337; C11B 1/00

(52) U.S. Cl. 424/731; 514/283; 514/449; 554/8

(57) ABSTRACT

The invention relates to a stabilised pharmaceutical composition comprising a pharmaceutically active substance poorly soluble in water, a solubilising agent with a low content of both basic and acidlic compounds and a polar organic solvent, in particular a stabilised injection concentrate, methods for preparing such stabilised pharmaceutical compositions and the use of a solubilising agent with a low content of both basic and acidic compounds to stabilise pharmaceutical compositions for pharmaceutically active substances.

The invention relates to a stabilised pharmaceutical composition comprising a pharmaceutically active substance poorly soluble in water, a solubilising agent with a low content of both basic and acidic compounds and a polar organic solvent, in particular a stabilised injection concentrate, methods for preparing such stabilised pharmaceutical compositions and the use of a solubilising agent with a low content of both basic and acidic compounds to stabilise pharmaceutical compositions for pharmaceutically active substances.

For the preparation of pharmaceutical compositions a suitable solvent or carrier system is required in order to disperse the pharmaceutically active agent so that the composition can be administered to a patient. The solvent must be capable of solubilizing or dispersing a therapeutically effective amount of the active agent to produce an effective composition. However, many pharmaceutically active compounds are not sufficiently soluble in solvents such as water. Another problem is that numerous pharmaceutical agents are unstable after dilution to infusion solutions or they exhibit degradation and loss of activity during storage in solvent systems. The low solubility and the proneness to degradation substantially limit the use of these pharmaceutically active compounds in actual therapy.

Examples of pharmaceutically active compounds which are poorly soluble in water and prone to degradation during storage are the antineoplastic agents paclitaxel and camptothecin derivatives.

To overcome the limitations of the solvent, in particular water, to solubilize pharmaceutically active agents, mixtures of two or more solvents are used. Such co-solvent systems comprise advantageously non-ionogenic solubilisers in combination with a suitable polar solvent. Such combined systems assure sufficient solubility of otherwise poorly water-soluble active agents both in liquid concentrates for injection and in infusion solutions obtained after dilution of the former. In such combined systems, ethanol is used frequently as the polar solvent and polyoxyethylated castor oils as solubiliser. A polyoxyethylated castor oil of standard quality is commercially available from BASF under the trade mark Cremophor EL. Cremophor EL is chemically a polyoxyethylated glycerol triricinoleate. A particular useful co-solvent system is a 50:50 mixture of ethanol and Cremophor EL, which can used for many active substances including paclitaxel or camptothecin derivatives that are poorly soluble in water.

The use of Cremophor EL as solubilizing agent in pharmaceutical compositions is associated with certain advantages, as shown by the patent application WO 91/02531. The document describes that Cremophor EL is capable of reversing the multi-drug resistance phenotype of a tumour cell line without altering the drug sensitivity of the parent cell line and can support haemopoiesis. Therefore Cremophor/ethanol systems are in particular suitable for the preparation of pharmaceutical compositions, in particular those formulated for the treatment of oncologic diseases.

Furthermore, possible undesirable adverse responses of an organism to Cremophor can easily be avoided by a pre-medication with steroids and antagonists of H₁ and H₂-receptors.

However, a main disadvantage of Cremophor EL is the high content of basic compounds. The basic impurities of Cremophor EL result in a continuously reduced stability and deteriorating quality of the pharmaceutical compositions until expiration date, whereby the content of active substance decreases and the content of potentially toxic decomposition products of the active substance and other components of the composition increases. Therefore, a number of recent patent documents relate to methods for stabilising pharmaceutical compositions comprising paclitaxel and polyoxyethylated castor oil.

Patent applications WO 94/12030 and WO 94/12031 disclose pharmaceutical compositions comprising paclitaxel and a polyoxyethylated castor oil such as Cremophor EL which are stabilised by the adjustment of the pH of the composition to a value of less than 8.1. For the adjustment of pH inorganic acids, e.g. hydrochloric acid, sulphuric acid, nitric acid, or low molecular organic acids, advantageously acetic acid or citric acid are used. The stabilising effects of acids are shown by a comparison of otherwise identical compositions. The content of taxol in a composition formulated with Cremophor EL, but without an acid (pH of 9.1) was 86,7% after 7 days at 40° C. In contrast, the content of taxol in a composition, whose pH was adjusted by the addition of citric acid to 6.2, was 96,6% after 7 days. A composition whose pH was adjusted by the addition of acetic acid to 6.7, exhibited a taxol content of 97,5% after 7 days.

Patent application WO 94/12198 discloses a pharmaceutical composition comprising taxol, a solubilising agent, advantageously a polyoxyethylated castor oil, an organic solvent, advantageously ethanol, and an acid that adjusts the pH of the composition to a value of less than 8.1. The pH can be adjusted essentially by the same acids as disclosed in the above mentioned patent applications WO 94/12030 and WO 94/12031.

EP 0 645 145 B1 describes a solvent system suitable for preparing a stabilised composition comprising a pharmaceutical compound. The solvent system comprises ethanol and a non-ionic solubilising agent, such as a polyoxyethylated oil, treated to reduce the carboxylate anion content to a sufficiently low concentration in order to minimize the decomposition of the pharmaceutical agent. The co-solvent system described is particularly suitable for use with pharmaceutical compounds such as paclitaxel, camptothecin and derivatives thereof that exhibit decomposition which is catalysed by carboxylate anions. According to this document the carboxylate anion content of the solvent can be reduced by passing the polyoxyethylated oil such as Cremophor EL through a chromatography column of aluminium oxide, whereby aluminium oxide efficiently adsorbs the carboxylate anions. The carboxylate content can also be reduced by the addition of an acid, such as a strong mineral acid. Pharmaceutical compositions of paclitaxel containing processed Cremophor EL and thus having a reduced carboxylate anion content are more stable than compositions containing unprocessed Cremophor EL.

U.S. Pat. No. 5,925,776 describes polyethoxylated castor oil with a low cation content, along with methods for lowering the cation content in polyethoxylated castor oil. The cations of interest, for example Al³⁺, K⁺, Ca²⁺ and Na⁺, can be removed by a pretreatment of the polyethoxylated castor oil, which is preferably Cremophor EL, with a strong cationic exchange resin. The low cationic content polyethoxylated castor oil can be utilized to prepare formulations of agents which were found to be sensitive to commercially available polyethoxylated castor oil, such as diclofenac and paclitaxel. Formulations of such agents prepared with low cationic content polyethoxylated castor oil are found to have improved stability against active agent degradation. However, a main disadvantage of the method described is based on the fact that there is a risk that the strong ion-exchange resin used to lower the cation content, can lead to a partial decomposition (splitting) of the polyoxyethylated castor oil, especially under the low pH conditions used, leading to an increased amount of free fatty acids.

Nowadays a processed polyoxyethylated castor oil is commercially available under the trade name Cremophor EL-P. Cremophor EL-P which has a lower content of basic compounds in comparison to Cremophor EL can be used for the preparation of a relatively stable composition of paclitaxel.

It is apparent from the prior art that the content of basic compounds in polyoxyethylated castor oil, particularly of carboxylate anions, is one of the main reasons for the instability of paclitaxel and similar antineoplastic compounds in formulations based on polyoxyethylated castor oil. All the relevant procedures solve, in different ways, the same problem, namely to decrease the content of basic compounds in the polyoxyethylated castor oil or in the final pharmaceutical composition comprising the polyoxyethylated castor oil. None of the prior art patent documents provides any procedure that could further improve the stability of the above-mentioned pharmaceutical substances in polyoxyethylated castor oil once the content of basic compounds was decreased to a value equal to or less than 0,6×10⁻⁶ gram equivalents/ml. Thus, there is a continuing need in the art for stabilised pharmaceutical compositions comprising an active agent which is poorly soluble in water.

Thus, the technical problem underlying the present invention is to provide a stabilised pharmaceutical composition which is especially suited for pharmaceutically active agents such as paclitaxel or camptothecin which shows a further improved stability in comparison to pharmaceutical compositions described in the prior art and prevents more efficiently the degradation of the active substance.

The present invention solves the technical problem by providing a stabilised pharmaceutical composition comprising a pharmaceutically active substance and a solvent system comprising a polar organic solvent and a solubilising agent, wherein the agent is a polyoxyethylated castor oil, characterised in that both the content of basic compounds is less than 0,6×10⁻⁶ gram equivalents/ml and the content of acidic compounds is equal to or less than 0,06 mass % based on the mass of the polyoxethylated castor oil. The present invention also solves the technical problem by providing methods for preparing stabilised pharmaceutical compositions whereby polyoxyethylated castor oil used as solubilising agent is pre-treated with an adsorbent in order to reduce the content of polar impurities, especially the content of acidic substances, and by the use of a solubilising agent with a low content of basic compounds and a low content of acidic compounds to stabilise a pharmaceutical composition comprising a pharmaceutically active substance which is poorly soluble in water.

Contrary to the existing technical prejudice, that the stability of pharmaceutically active compounds such as paclitaxel or camptothecin in a co-solvent system comprising a polar organic solvent and a solubiliser, such as a polyoxyethylated castor oil, depends only on the presence of basic compounds, the inventors of the present invention have surprisingly found that acidic compounds present in the composition, in particular in the solubilizing agent, also have a great impact on the stability of such pharmaceutically active compounds.

For example, the commercially available Cremophor EL-P characterized by a reduced content of basic compounds, however, contains impurities such as fatty acids and the oxyethylated forms thereof, polyethylenglycol diricinoleate and small amounts of corresponding free glycols. Upon treatment of Cremophor EL-P with a suitable adsorbing agent to remove these acidic compounds, pharmaceutical compositions comprising such pre-treated Cremophor EL-P exhibit a greatly enhanced stability of the pharmaceutically active agents in comparison with compositions prepared with untreated Cremophor EL-P as shown by the determination of decomposition products of pharmaceutically active compounds in the compositions formed after a long-term storage.

Thus, the analysis of pharmaceutical compositions of paclitaxel prepared with treated or untreated Cremophor EL-P has revealed that in compositions with treated Cremophor EL-P after 3 month storage the amounts of the main decomposition products such as baccatin III, 10-deacetyl-paclitaxel, 10-deacetyl-7-epi-paclitaxel, 7-epi-paclitaxel and cephalomannin are much smaller than in compositions prepared with untreated Cremophor EL-P. Also, injections of camptothecin prepared with treated Cremophor EL-P exhibit an improved stability, since after 14 days these injections contain more nondecomposed camptothecin than camptothecin injections prepared with untreated Cremophor EL-P.

In summary, these results obtained according to the invention confirm that also acidic compounds present in the solubilizing agent contribute to the decomposition of active agents such as paclitaxel or camptothecin. The less the content of both basic and acidic compounds in the composition with a polyoxyethylated castor oil, the more stable the pharmaceutically active compound in it.

Therefore, according to the invention a further improvement of the stability of pharmaceutically active compounds in a co-solvent system comprising a polar organic solvent and a solubiliser, in particular a polyoxyethylated castor oil with a low content of basic compounds such as Cremophor EL-P, can be obtained by decreasing the content of acidic compounds to a value equal to or less than 0.06 mass % based on the mass of the polyoxyethylated castor oil. According to the invention in particular the content of free fatty acids such as ricinoleic acid, oleic acid and palmitic acid must be less than 0.06 mass %.

The inventive stabilised pharmaceutical composition is in particular suited for pharmaceutically active agents which exhibit degradation and loss of activity during storage, e.g. paclitaxel and camptothecin and derivatives thereof. According to the invention the formation of some decomposition compounds known can be caused not only by basic components of the solubilizing agent but also by acidic components thereof.

The present invention therefore relates to a pharmaceutical composition with improved stability comprising a pharmaceutically active compound and a solvent system comprising a polar organic solvent and a solubiliser, wherein the solubiliser is a polyoxyethylated castor oil said composition being characterised by a low content of basic compounds, particularly carboxylate anions, equal to or less than 0,6×10⁻⁶ gram equivalents/ml and by a low content of acidic compounds which is equal to or less than 0.06 mass % based on the mass of the polyoxyethylated castor oil.

According to the invention, the term “pharmaceutical composition” refers to a mixture of substances which is used for diagnostic, therapeutic or prophylactic purposes, that is which supports or restores the health of a human or animal body, and which comprises at least a natural or synthetically generated active agent, inducing the desired therapeutic effect. The pharmaceutical composition can comprise one or more pharmaceutically acceptable excipients and additives usually employed in the art. According to the invention, a pharmaceutical composition which is “stabilised” or which has “improved stability” is a composition in which the decomposition of the active agent is prevented or at least delayed, so that even after long term storage more than 90%, in particular more than 95%, preferably more than 97%, most preferably more than 99% of the active agent have not undergone a decomposition. In a particular preferred embodiment of the invention the stabilised pharmaceutical composition has the form of an injection comprising a pharmaceutically active agent. “Injections” are sterile liquid dosage forms including solutions, suspensions or emulsions for parenteral administration. Such liquid dosage form may also contain preserving, wetting, emulsifying and dispersing agents. Injections may be sterilized by, for example, filtration through a bacteria and/or other pathogens retaining filters, by incorporating sterilising agents into the compositions and/or by irradiating the compositions. They can also be manufactured using sterile components immediately before use.

The term “pharmaceutically active agent” or “active agent” refers to any compound or derivate thereof which can affect or recognise biological cells or parts thereof, in particular cell organelles or cellular components, by an direct or indirect interaction with cellular macromolecules whereby a number of functional changes is induced leading to a biological effect in the body. In particular, such active agents are diagnostics or therapeutics. In the context of the present invention the term “active agents” or “pharmaceutically active agents” refers in particular to therapeutics, i.e. substances which can be administered as a preventive measure or during the course of a disease, disorder or condition to organisms in need of such a treatment in order to prevent or to reduce or to abolish a disease, disorder or condition.

In a preferred embodiment of the invention, the stabilised pharmaceutical composition, in particular injection, comprises an pharmaceutically active agent that is poorly soluble in water and/or sensitive towards degradation during storage. According to the invention the pharmaceutically active agent is preferably selected from the group consisting of paclitaxel, camptothecin and derivatives thereof.

Therefore, in a particular preferred embodiment of the invention the pharmaceutical composition comprises paclitaxel as pharmaceutically active agent.

Paclitaxel is an pharmaceutically active agent with antineoplastic activity, which is commercially available from Bristol-Myers-Squibb under the trade name TAXOL. Paclitaxel is believed to function as a mitotic spindly poison and as a potent inhibitor for cell replication. Paclitaxel is a compound of formula (I):

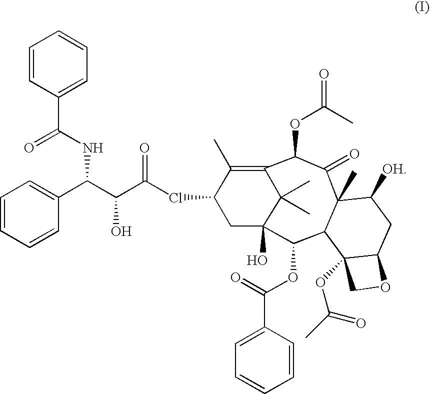

During storage the main products of paclitaxel decomposition are baccatin III, 10-deacetylpaclitaxel, 10-deacetyl-7-epi-paclitaxel, 7-epipaclitaxel and cephalomannin. As known in the art, the formation of these decomposition products of paclitaxel can be catalysed by basic compounds. According to the invention the formation of these paclitaxel decomposition products can also be catalysed by acidic compounds.

In another preferred embodiment of the invention the pharmaceutical composition comprises camptothecin as pharmaceutically active agent.

Camptothecin is a pharmaceutically active substance of formula (II):

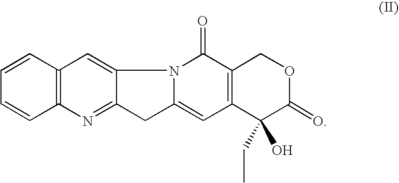

Camptothecin and derivatives thereof (irinotecan, topotecan etc.) also exhibit an important antineoplastic activity. The therapeutic activity of these compounds is conditioned by the existence of a closed lactone ring of the given structure. The lactone ring may be split by solvolysis into an open-chain carboxyl form, said form however being far less therapeutically effective. Such a solvolysis can be induced by both bases and acids present in the composition, in particular in the solubilizer.

The term “derivative thereof” refers to non-toxic functional equivalents or derivatives of paclitaxel or camptothecin, which can be obtained by substitution of atoms or molecular groups or bonds of the paclitaxel or camptothecin molecule, whereby the basic structure is not changed, and which differ from the structure of paclitaxel or camptothecin in at least one position.

The term “solvent” refers to an inorganic or organic fluid in which another liquid or solid compound can be solved. A prerequisite of an solvent is that neither the solvent nor the substance to be solved undergo an chemical change. A physical precondition for an solvent is the presence of polar or non-polar residues. A “polar organic solvent” therefore refers to an organic solvent with polar residues.

In a preferred embodiment of the invention the polar organic solvent is ethanol.

The term “solubiliser” or “solubilizing agent” refers to substances which render a compound which is poorly soluble or insoluble in a certain solvent, soluble or emulsifiable in that solvent. Optionally a solubiliser can be a surface active agent. An example of an solubilizer is polyoxyethylated castor oil. Polyoxyethylated castor oil, for example Cremophor EL, is chemically a polyoxyethylated glycerol triricinoleate. Cremophor EL is characterized by the big content of basic compounds, in particular carboxylate anions, which affect the stability of pharmaceutical compositions.

According to the invention the polyoxyethylated castor oil used as solubilizer has a low content of basic compounds, such as carboxylate anions, of less than 0,6×10⁻⁶ gram equivalents/ml. In a particular preferred embodiment of the invention the polyoxyethylated castor oil with such a low content of basic compounds, used as solubilizer, is Cremophor EL-P prepared according to prior art. As impurities, the product contains fatty acids and its oxyethylated forms, polyethylenglycol diricinoleate and small amounts of corresponding free glycols. However, Cremophor EL-P has a high content of free fatty acids, in particular C₁₂ to C₁₈ fatty acids of equal to or less than 1.0%. Cremophor EL-P comprises approx. 0,2% ricinoleic acid and approximately 0.1% oleic acid and 0.1% palmitic acid. The amount of ricinoleic acid of 0.2% corresponds to approximately 50% of the stoichiometric amount relative to paclitaxel present in the composition and is therefore relatively high.

In a more preferred embodiment of the invention the content of free fatty acids is less than 0.06 mass % based on the mass of the polyoxyethylated castor oil. In another particular preferred embodiment of the invention the content of both free oleic acid and palmitic acid must be equal to or less than 0,01 mass % based on the mass of the polyoxyethylated castor oil.

A concentration of carboxylate anions of less than or equal to 0,6×10⁻⁶ gram equivalents/ml can be determined as specified in U.S. Pat. No. 5,504,102, in particular by indirect measurement by adding acid, in particular HCl. A direct measurement of fatty acids is possible by the GC method after derivatisation of these compounds.

According to the invention the content of acidic compounds in the composition, in particular in solubilizer of the co-solvent system, can be lowered by a number of methods.

In a preferred embodiment advantageously the content of polar impurities, in particular acidic compounds in the solubilizer, i.e. the polyoxyethylated castor oil, is reduced by treatment of the polyoxyethylated castor oil with an adsorbent. According to the present invention an “adsorbent” is usually a solid substance, which is capable of selectively enriching certain components of a mixture at its boundary surface due to its large surface.

A preferred embodiment of the present invention therefore relates to a stabilised pharmaceutical composition wherein the polyoxyethylated castor oil is a polyoxyethylated castor oil treated with an adsorbent. Preferably, the adsorbent used to reduce the content of acidic compounds is silica gel or aluminosilicate. In a particular preferred embodiment of the invention the polyoxyethylated castor oil is Cremophor EL-P having a low content of basic compounds and treated with silica gel (5 to 10 mass %) at a moderate temperature, preferably in the range of 40 to 60° C., in particular 50° C. Silica gel is a slightly acidic and polar adsorbent which eliminates polar impurities including acidic compounds in a simple and effective way. By the treatment of polyoxyethylated castor oils, such as Cremophor EL-P, with such an adsorbent the content of acidic compounds can easily be reduced to amounts of less than 0,06%.

The present invention relates also to methods for preparing stabilised pharmaceutical compositions comprising a pharmaceutically active substance in a solvent system comprising a polar solvent and a solubilising agent, wherein the solubilising agent is a polyoxyethylated castor oil, comprising the steps of treating a polyoxyethylated castor oil with a low content of a basic compound with an adsorbent in order to reduce the content of polar impurities and mixing the treated polyoxyethylated castor oil with a certain amount of the polar orgainic solvent and a certain amount of the pharmaceutically active substance.

In a preferred embodiment the polyoxyethylated castor oil to be treated comprises basic compounds in a content of less than 0,6×10⁻⁶ gram equivalents based on the mass of the polyoxyethylated castor oil. In a particular preferred embodiment the polyoxyethylated castor oil with a reduced amount of basic compounds to be treated is Cremophor EL-P.

According to the invention the polyoxyethylated castor oil with a low or reduced content of a basic compound is treated with an adsorbent to reduce the content of polar impurities, in particular acidic substances such as free fatty acids.

In a preferred embodiment of the inventive method, the adsorbent used to reduce the amount of acidic substances is aluminosilicate or silica gel. In a particularly preferred embodiment Cremophor EL-P is treated with silica gel (5 to 10 mass %) at a moderate temperature, preferably in the range of 40 to 60° C., in particular 50° C. After treatment with the adsorbent Cremophor EL-P has advantageously a content of acidic compounds of equal to or less than 0,06 mass % based on the mass of the polyoxyethylated castor oil. Preferably Cremophor EL-P comprises ricinoleic acid in a content of equal to or less than 0,05 mass % and oleic acid and palmitic acid in a content of equal to or less than 0,01 mass % based on the mass of the polyoxyethylated castor oil.

In a preferred embodiment of the inventive method the polar solvent used for the preparation of the stabilised pharmaceutical composition is ethanol. The polyoxyethylated castor oil treated by the adsorbent is preferably mixed with ethanol in a ratio of 1:1.

In another preferred embodiment of the inventive method, the pharmaceutically active substance is poorly soluble in water. In particular, the pharmaceutically active substance is selected from the group consisting of paclitaxel, camptothecin and their derivatives.

Another aspect of the invention relates to the use of a solubilising agent with a low content of basic compounds and a low content of acidic compounds to stabilise a pharmaceutical composition comprising a pharmaceutically active substance which is poorly soluble in water. In a preferred embodiment the solubilising agent is polyoxyethylated castor oil with a low content of basic compounds, in particular of less than 0,6×10⁻⁶ gram equivalents based on the mass of the polyoxyethylated castor oil, which is treated with an adsorbent to reduce the amount in particular of the acidic compounds such as free fatty acids, in particular ricinoleic acid, oleic acid and palmitic acid. In a particular preferred embodiment the polyoxyethylated castor oil comprising a low content of basic compounds and treated with the adsorbent is Cremophor EL-P. Preferably, the polyoxyethylated castor oil treated with the adsorbent such a silica gel or aluminosilicate and used for stabilising pharmaceutical compositions has a content of acidic compounds less than 0,6 mass % based on the mass of the polyoxyethylated castor oil. In another embodiment the solubilising agent with a low content of basic compounds and a low content of acidic compounds is used to stabilise pharmaceutical compositions wherein the pharmaceutically active substance is selected from the group consisting of paclitaxel, camptothecin and their derivatives.

The invention will be further explained by the following examples. The examples are for illustrative purposes only and shall by no means limit the scope of the invention.

### EXAMPLES

### Example 1

### Description of Analytical Methods

1. Determination of the Content of Free Fatty Acids in Polyoxyethylated Castor Oil by GC Method

The content of free fatty acids was determined by the BASF test method 0330/01e. Free fatty acids in polyoxyethylated castor oil were converted to volatile silylester compounds by means of N-methyl-N-trimethylsilyltrifluoracetamide. Volatile silylester compounds were analysed by the GC method (capillary column HP-5; 30 m; 0.32 mm ID; 0.25 μm; FID detector). The content was quantified by internal standard method with methylmargarate.

The content of free fatty acids in Cremophor EL-P, as determined by the BASF GC method was as follows:
Ricinoleic acid 0,07%
oleic acid 0,02%
palmitic acid 0,02%.
   2. Determination of the Content of Paclitaxel and Related Compounds in the Composition by HPLC

The standard HPLC method described in Pharmacopoeial Forum, Vol.24, No.6, Nov.-Dec. 1998, p.7167 was used.

### EXAMPLE 2

### Preparation of Cremophor EL-P with low Acidic Compound Content (LAC)

Starting Materials:

Cremophor EL-P(BASF): water content 0,3%; pH of 10% aqueous extract 6,3; total content of free fatty acids: 0,18%;
Silica gel: Kieselgel 60, 0.063-0.200 mm
Cremophor EL-P (3 kg) and 5 mass % of silica gel were stirred under dry nitrogen 2 hours at 50° C. Cremophor was then filtered. This process was repeated once again. The yield of Cremophor EL-P-LAC was almost 90%. The total content of free fatty acids in Cremophor EL-P-LAC was 0.06 mass %, the content of ricinoleic acid was 0.05 mass %, the content of oleic acid and palmitic acid was less than 0.01 mass % respectively. A 10% solution of Cremophor EL-P-LAC in water had a pH value of 6,3. This shows that the removal of fatty acids did not alter the pH value in Cremophor in comparison to Cremophor EL-P.

### EXAMPLE 3

### Preparation of Paclitaxel Injections

Starting materials:
Ethanol: water content <0,1%,
As solubilising agents Cremophor EL-P (BASF) and Cremophor EL-P from Example 2 were used.

Cremophor EL-P-LAC: Cremophor EL-P-LAC from Example 2 was used.

Paclitaxel API (by SUAN Pharma): paclitaxel, content 99,73 mass % (determined by high performance liquid chromatography).

Under GMP conditions, a solution of Cremophor and ethanol in volume ratio 1:1 was prepared, with a paclitaxel concentration of 6 mg/ml of the solution. The resulting solution was filtered under sterile conditions through a filter with a porosity of 0,2 mm. Volumes of 5 ml were filled into glass vials for antibiotics of the first hydrolytic class. The vials were closed under nitrogen atmosphere with Omniflex rubber stoppers and aluminium seal.

### EXAMPLE 4

### Stability Study of Paclitaxel Compositions

The stability study was performed by subjecting the injections to a temperature of 40° C. at 75% R.H. for three months. The compositions were analysed by validated HPLC method. The results are summarised in Table 1.

TABLE 1
The three months stability study of paclitaxel
injections at 40° C. and 75% R.H.

| | | | injections |
|---|---|---|---|
| | | injections | Cremophor |
| | | Cremophor EL-P | EL-P-LAC |
| Compound | Time 0% | 3 months % | 3 months % |
| Paclitaxel | 99.66 | 98.27 | 99.52 |
| Baccatim III | non detectable | 0.09 | non detect |
| 10-deacetyltaxel | non detectable | 0.35 | 0.07 |
| 7-epi-taxel | 0.03 | 0.10 | 0.07 |
| 7-epi-10-deacetyltaxel | 0.01 | 0.04 | 0.03 |
| Cephalomannin | 0.15 | 0.29 | 0.20 |
| unknown impurities | 0 | 2 | 0 |
| above 0.1% | | | |
| total relative | 0.34 | 1.73 | 0.48 |
| impurities | | | |

The compositions of both injections were practically identical in time 0.

The results in Table 1 demonstrate the superior stability of paclitaxel in the composition with polyoxyethylated castor oil and ethanol according to the present invention, characterised by low contents of both basic and acidic compounds. Baccatin III is the main impurity from basic splitting and is negligible in both injections. The results show, however, that the formation of undesired 10-deacetyltaxel, 7-epi-taxel, cephalomannin and at least two unknown impurities is supported by the presence of free acids in compositions, since compositions comprising Cremophor EL-P-LAC show reduced amounts of these compounds after 3 months.

Pharmaceutical composition based on a polyoxyethylated castor oil with low content of both basic and acidic compounds according to present invention may be used not only for paclitaxel, but also for other pharmaceutically active compounds that are poorly soluble in water and/or susceptible to a degradation during storage. For instance, the composition of the invention can also be applied to pharmaceutical compositions of camptothecin and derivatives thereof as shown in the following Example 5.

### EXAMPLE 5

### Preparation of Camptothecin Compositions

In this example Cremophor EL-P and Cremophor EL-P-LAC from Example 2 were used as solubilising agents. Under GMP conditions, 600 mg of 7-ethyl-10-hydroxycamptothecin (purity 99.2 mass % as determined by high performance liquid chromatography) were dissolved in 50 ml of ethanol at 50° C. The solution was cooled to 21° C. and 50 ml of the respective Cremophor was added to the composition. The resulted solution was filtered under sterile conditions through a filter of 0.2 μm porosity and was filled in 5 ml volumes into glass antibiotic vials of 1st hydrolytic class. The vials were closed under nitrogen atmosphere with Omniflex rubber stoppers and aluminium seal.

The stability study of injections was performed by subjecting them to a temperature of 50° C. and 75% R.H. for 14 days. The content of 7-ethyl-10-hydroxycamptothecin in the injections was determined by a standardised method of high performance liquid chromatography. The obtained results are shown in the following Table 2.

TABLE 2
14 days stability study of 7-ethyl-10-
hydroxycamptothecin injections at 50° C. and 75% R.H.

| | 7-ethyl-10-hydroxycamptothecin |
|---|---|
| Injections | content, % |
| Injections from Cremophor EL-P-LAC | 98.2 |
| Injections from Cremophor EL-P | 97.2 |

It is apparent from the results presented that the invention may also be advantageously used for providing stable pharmaceutical compositions comprising camptothecin and/or derivatives thereof as an active substance.

1. Stabilised pharmaceutical composition comprising a pharmaceutically active substance and a solvent system comprising a polar organic solvent and a solubilising agent, wherein the agent is a polyoxyethylated castor oil treated with an absorbent, namely silica gel or aluminosilicate to reduce the content of polar impurities including acidic compounds, characterised in that both the content of basic compounds is less than 0.6×10⁻⁶ gram equivalents/ml and the content of acidic compounds is less than 0.06 mass % based on the mass of the polyoxyethylated castor oil.

2. Stabilised pharmaceutical composition according to claim 1, wherein the polar organic solvent is ethanol.

3. Stabilised pharmaceutical composition according to claim 1, wherein the pharmaceutically active substance is an active substance which is poorly soluble in water and/or sensitive to degradation during storage.

4. Stabilised pharmaceutical composition according to claim 3, wherein the pharmaceutically active compound is selected from the group consisting of paclitaxel, camptothecin and their derivatives.

5. Method for preparing a stabilised pharmaceutical composition comprising a pharmaceutically active substance in a solvent system comprising a polar solvent and a solubilising agent, wherein the solubilising agent is a polyoxyethylated castor oil comprising the steps of treating a polyoxyethylated castor oil, which comprises basic compounds in a content of less than 0.6×10⁻⁶ gram equivalents based on the mass of the polyoxyethylated castor oil with an adsorbent, namely silica gel or aluminosilicate, in order to reduce the content of polar impurities, including acidic substances, whereby the content of acidic substance is reduced to less than 0.06 mass % based on the mass of polyoxyethylated castor oil and mixing the treated polyoxyethylated castor oil and mixing the treated polyoxyethylated castor oil with an amount of the polar organic solvent and an amount of the pharmaceutically active substance.

6. Method according to claim 5, wherein the polyoxyethylated castor oil is Cremophor EL-P.

7. Method according to claim 5 wherein the polar solvent is ethanol.

8. Method according to claim 7, wherein the treated polyoxyethylated castor oil is mixed with ethanol in a ratio of 1:1.

9. Method according to claim 5, wherein the pharmaceutically active substance is poorly soluble in water.

10. Method according to claim 9, wherein the pharmaceutically active substance is selected from the group consisting of paclitaxel, camptothecin and their derivatives.

11. Use of a solubilising agent with a content of basic compounds and a content of acidic compounds to stabilise a pharmaceutical composition comprising a pharmaceutically active substance which is poorly soluble in water, wherein the solubilising agent is polyoxyethylated castor oil with a content of basic compounds of less than 0.6×10⁻⁶ gram equivalents based on the mass of polyoxyethylated castor oil treated with an absorbent to reduce the amount of acidic compounds, wherein the absorbent used to reduce the acidic compounds is silica gel or aluminosilicate and wherein the polyoxyethylated castor oil has a content of acidic compounds less than 0.06 mass % based on the mass of the polyoxyethylated castor oil.

12. Use according to claim 11, wherein the polyoxyethylated castor oil is Cremophor EL-P.

13. Use according to claim 11, wherein the pharmaceutically active substance is selected from the group consisting of paclitaxel, camptothecin and their derivatives.

14. Solubilising agent which is a polyoxyethylated castor oil treated with an adsorbent, namely silica gel or aluminosilicate to reduce the content of polar impurities including acidic compounds, characterised in that both the content of basic compounds is less than 0.6×10⁻⁶ gram equivalent/ml and the content of acidic compounds is less than 0.6 mass % based on the mass of the polyoxyethylated castor oil.
